(19) United States
(12) Patent Application Publication
(10) Pub. No.: US 2009/0011516 A1
(43) Pub. Date: Jan. 8, 2009

(54) Methods and Assays for the Detection of Nitrogen Uptake by a Plant and Uses Thereof

(75) Inventors: Dale F. Loussaert, Clive, IA (US); Mary J. Frank, Des Moines, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL, INC., Johnston, IA (US)

(21) Appl. No.: 12/166,473

(22) Filed: Jul. 2, 2008

Related U.S. Application Data
(60) Provisional application No. 60/947,726, filed on Jul. 3, 2007.

(51) Int. Cl. G01N 33/00

(52) U.S. Cl. 436/114

(57) ABSTRACT

The invention provides a rapid and efficient method and assay for monitoring nitrogen uptake by a plant using a pH indicator. The plant is exposed to medium comprising one or more sources of nitrogen, such as nitrate or ammonia, and a pH indicator. The plant is exposed to the source of nitrogen for a time sufficient for it to be taken up by the plant. As nitrate is taken up from the medium, the medium becomes more basic, that is the pH increases. Conversely, as ammonia is taken up from the medium, the medium becomes more acidic and the pH decreases. The change in the pH of the medium may be optically detected and correlated to the amount of nitrate or ammonia remaining in the medium. Accordingly, the amount of nitrate or ammonia taken up by the plant or remaining in the medium may be determined.

### CROSS REFERENCE

This utility application claims the benefit U.S. Provisional Application No. 60/947,726, filed Jul. 3, 2007 which is incorporated herein by reference.

### BACKGROUND OF THE INVENTION

Increased nitrogen use efficiency can result from enhanced uptake and assimilation of nitrogen fertilizer and/or the subsequent remobilization and reutilization of accumulated nitrogen reserves. Plants containing genes that render them more productive with current fertilizer application standards, or maintaining their productive rates with significantly reduced fertilizer input can therefore be used for the enhancement of yield. Improving the nitrogen use efficiency in maize and other plants would increase yields per unit of input nitrogen fertilizer, both in developing nations where access to nitrogen fertilizer is limited and in developed nations were the level of nitrogen use remains high. Nitrogen utilization improvement also allows decreases on-farm input costs, decreased use and dependence on the non-renewable energy sources required for nitrogen fertilizer production, and decreases the environmental impact of nitrogen fertilizer manufacturing and agricultural use.

Although a variety of techniques have been developed to measure nitrogen uptake in plants, they generally are inconvenient and require several weeks of plant growth prior to measurement. For example, conventional methods for determining nitrogen uptake employ an indirect approach in measuring the amount of nitrate taken up by a plant using a chlorophyll meter, such as a spadmeter, in the field to estimate the green color of a plant or by measuring the plant's biomass or dry weight as a surrogate to nitrogen concentration. Thus, these approaches lack a high throughput method that provides rapid information on nitrogen uptake for multiple plants early in their development in an efficient manner in a convenient laboratory setting. For these and other reasons, there is a need for the present invention.

### BRIEF SUMMARY OF THE INVENTION

The invention provides a rapid and efficient method and assay for monitoring nitrogen uptake by a plant using a pH indicator. The plant is exposed to medium comprising one or more sources of nitrogen, such as nitrate or ammonia, and a pH indicator. The plant is exposed to the source of nitrogen for a time sufficient for it to be taken up by the plant. As nitrate is taken up from the medium, the medium becomes more basic, that is the pH increases. Conversely, as ammonia is taken up from the medium, the medium becomes more acidic and the pH decreases. The change in the pH of the medium may be optically detected using any number of methods and correlated to the amount of nitrate or ammonia remaining in the medium. Accordingly, the amount of nitrate or ammonia taken up by the plant or remaining in the medium may be determined. The differences in change of pH or amount of nitrogen taken up by a plant may be compared to one or more other plants to determine which plant has the greater nitrate or ammonia uptake efficiency. The methods and assays of the present invention may be used to screen and identify polynucleotides that modulate or are suspected of modulating nitrate or ammonia uptake.

### BRIEF DESCRIPTION OF THE DRAWINGS

FIG. 1. Determination of nitrate remaining in medium with respect to color of pH indicator dye. As the medium becomes more basic, the medium containing bromophenol red will change from yellow to peach to pink. Nitrate analysis of the medium shows that more nitrate is remaining in the medium when the color of medium is yellow than when the medium is pink. Solid black bar indicates yellow medium, solid gray bar indicates peach colored medium and hatched bar indicates pink medium. Error bars indicate standard deviation.

FIG. 2. On the left is a depiction of the mechanism of nitrate uptake. As 1 mole of nitrate is absorbed 2 moles of acid are absorbed making the medium basic. On the upper right is a graft showing the absorption at 590 nM increases as base is added to the medium. In the lower right, the same relationship between addition of base and the increase in fluorescence of fluorescein is shown.

FIG. 3. The plant, pot and all, growing in TURFACE® MVP. (Profile Products LLC (Buffalo Grove, Ill.)) is put inside another container full of nutrient solution adjusted to pH 5 containing 100 μM bromocresol purple and allowed time with aeration for the plant to absorb nitrate and acid making the medium basic (pH=6+).

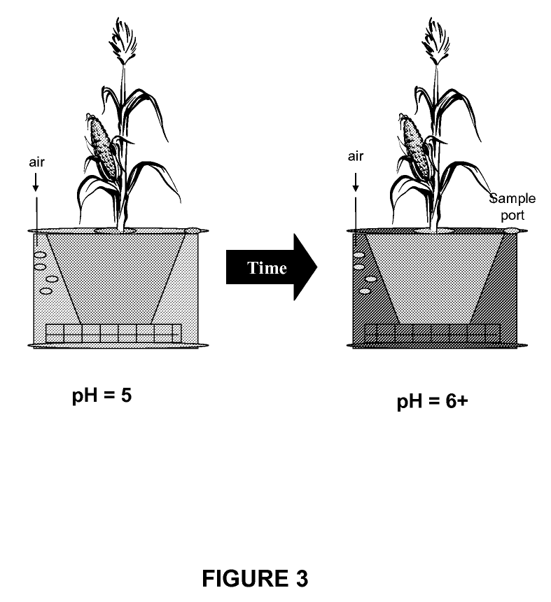

FIG. 4. As nitrate (solid squares) is removed from the medium, the pH increases as shown by the increase in absorption at 590 (open squares) and increased fluorescence (Ex 420, Em 530) of fluorescein (triangles).

FIG. 5. Plants with different capabilities to take up nitrate show different responses in the assay of the present invention. PN3394, a hybrid, and GS3/GF3×2, a backcross, take up nitrate at a much faster rate than the inbreds A63 and A188 and that difference is reflected in the slope of absorbance at 590 nM.

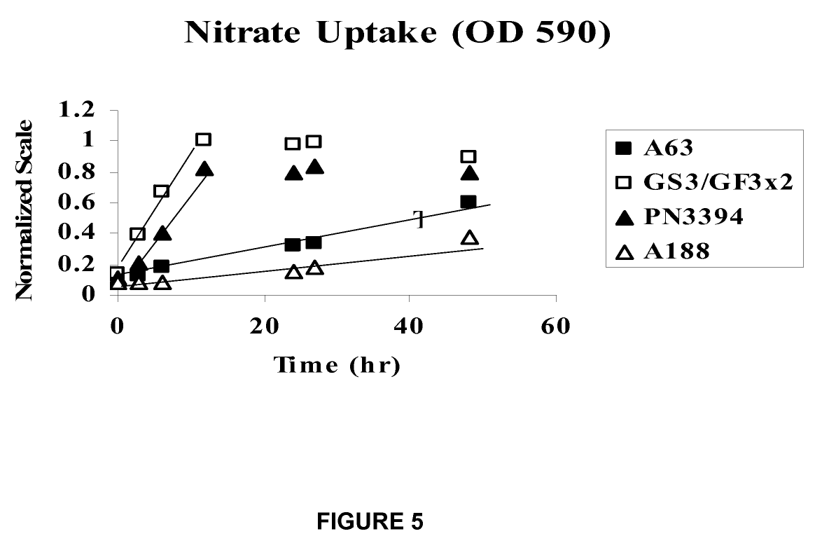

FIG. 6. Prophetic example of ammonia uptake. As NH4+ is absorbed (dashed line) the pH will drop (solid line). Thus if the pH was started at 6.5 (bromocresol purple is purple) and allowed to take up ammonia for a period of time the medium would become yellow (lower pH) and the absorption at 590 nM would decrease.

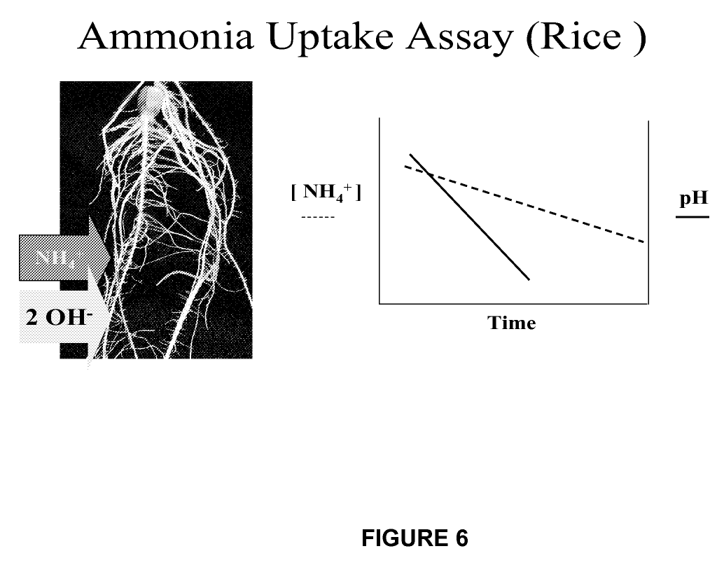

### DETAILED DESCRIPTION OF THE PREFERRED EMBODIMENT

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

In accordance with the present invention, methods and assays are described for the monitoring of nitrogen uptake, such as nitrate or ammonia, by plants, and for the identification of candidate polynucleotides that modulate nitrate or ammonia uptake. Without wishing to be bound by this theory, plants having polynucleotides that increase nitrate or ammonia uptake are believed to have increased yield and/or biomass. Methods and assays of the present invention use a pH indicator to monitor a plant's nitrate or ammonia uptake from medium. As nitrate is transported into the plant cell, two protons are also co-transported into the cell. As ammonium is transported into the plant cell, two hydroxide ions are also co-transported into the cell. Thus, the medium becomes more basic from nitrate leaving the medium and more acidic when ammonia is taken up from the medium, resulting in a change in pH which can be detected in any number of ways, for example, by optical density.

### DEFINITIONS

The term “source of nitrogen” refers to any form of nitrate or ammonia.

The term “medium”, as used herein, may include any medium, such as soil, agar, PHYTAGEL® agar substitute (Sigma-Aldrich, St. Louis, Mo.), water, nutrient solutions or any other medium or sampling of a medium that comes in contact with the plant and is capable of facilitating the growth of a plant.

The term “pH indicator” includes dyes that change optical properties, such as absorbance or fluorescence, with changes in pH. Any suitable pH indicator that is not phytotoxic may be used. One skilled in the art will be able to select the appropriate pH indicators for measuring acidic or basic changes of the medium.

The term “plant” includes but is not limited to a plant cell, a plant protoplast, plant cell tissue culture from which a plant can be regenerated, plant calli, a plant clump, and a plant cell that is intact in plants or parts of plants such as an embryo, seed, root, root tip, and the like.

The methods and assays of the present invention may be used to determine nitrogen uptake by any plant species of interest, including, but not limited to, monocots and dicots. Examples of plant species of interest include, but are not limited to, Arabdidopsis, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

A “subject plant or plant cell” refers to a plant that is being screened for nitrogen uptake.

A “control plant or plant cell” may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); or (c) a plant or plant cell which is a non-transformed segregant among progeny of a subject plant or plant cell. Thus, a “control plant” or “control plant cell” provides a reference point for measuring changes in phenotype of the subject plant or plant cell, for example, a difference in the change of pH in the medium or nitrogen concentration in the medium of the control plant as compared to the change of pH in the medium or nitrogen concentration in the medium of the subject plant.

A “control medium” provides a reference point for measuring changes in phenotype of the subject plant or plant cell, for example, a change in pH of the medium or nitrogen concentration in the medium. A “control medium” may comprise, for example: (a) the medium of a wild-type plant or cell, i.e., of the same genotype as the subject plant or cell; (b) the medium of a plant or plant cell of the same genotype as the starting material but which has been transformed with a null construct (i.e., with a construct which has no known effect on the trait of interest, such as a construct comprising a marker gene); or (c) the medium of a plant or plant cell which is a non-transformed segregant among progeny of a subject plant or plant cell.

In some cases, the “control plant” will be exposed to a “control medium” that has the same amount of the pH indicator as the “subject plant” but has varying amounts of nitrogen, for example, no nitrogen or the same amount of nitrogen as administered to the “subject plant”.

As used herein, “yield” may include reference to bushels per acre of a grain crop at harvest, as adjusted for grain moisture (15% typically for maize, for example), and the volume of biomass generated (for forage crops such as alfalfa, and plant root size for multiple crops). Grain moisture is measured in the grain at harvest. The adjusted test weight of grain is determined to be the weight in pounds per bushel, adjusted for grain moisture level at harvest. Biomass may be measured as the weight of harvestable plant material generated. One skilled in the art will be able to determine yield or biomass for a particular plant.

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

As used herein, the term “modulate”, “modulates” or “modulating” refers to a change, i.e. an increase or decrease in the level or amount of nitrate or ammonia that is taken up by the plant or remains in the medium of the plant.

According to the present invention, a method of monitoring nitrogen uptake of a plant includes contacting a plant with a pH indicator and a source of nitrogen for a time sufficient for the nitrogen to be taken up by the plant so that if a change in pH is to occur it is detectable.

Any plant may be screened using the methods and assays described herein, including but not limited to transgenics, inbreds, hybrids, and non-transformed plants. This also includes plants that have been treated with a mutagen, such as ethyl methanesulfonate (EMS) and the like. In one aspect, the plant to be screened includes a candidate polynucleotide suspected of modifying nitrogen uptake of the plant. In another aspect, the plant comprises individual or combinations of genes to be screened for their effect on nitrogen uptake.

In one aspect, the plant is grown in the presence of a source of nitrogen and a pH indicator for a period of time so that the source of nitrogen may be taken up by the plant. Such knowledge for determining the length of time for nitrogen uptake is well within the knowledge of one skilled in the art. For example, the medium or a sampling of the medium may be measured for a pH value at various time points, for example, at an initial starting point and then at various time points thereafter. Time points may vary from hours to days depending on the criteria of the experimental design and the type of plant being examined. Such criteria include but are not limited to the amount of nitrate or ammonium in the medium, the amount of medium, the pH indicator dye used and the type of plant being studied. For example, using the methods and assays described herein, nitrate uptake for maize plants and Arabidopsis plants may be determined in as little as a few hours or over several days respectively.

The source of nitrogen and the pH indicator may be administered to the medium of the plant simultaneously, separately, or consecutively with respect to one another. The plant may be exposed to a source of nitrogen in any suitable manner, including for example administering a solution having nitrate, such as potassium nitrate, to the medium. Any suitable form of nitrogen may be used, including, nitrate or ammonia or salts thereof, such as ammonium citrate or ammonium succinate. The source of nitrogen used may depend on the plant being screened for nitrogen uptake taking into consideration whether the plant assimilates ammonia ions as does rice or nitrate as does maize and other plants, such as crop plants.

In one aspect, the plant is exposed to a pH indicator, for example, by administering the pH indicator to the medium of the plant, e.g., immersion of the plants' medium in a solution comprising the pH indicator or introducing the pH indicator to the medium prior, during or after contact with the plant. In one aspect, the pH indicator may be administered to the medium by itself or in combination with other pH indicators, for example, the combination of bromocresol purple and fluorescein may be used in the methods and assays. The pH indicator may be administered in any suitable form or state, including but not limited to a liquid. The pH indicator may be dissolved in a solvent such as water or a nutrient solution. The pH indicator may be administered to the medium in the form of a salt, such as a sodium salt generated by reacting the indicator with sodium hydroxide.

The methods and assays may be conducted using an appropriate pH indicator that detects the occurrence of a change in acidity or alkalinity of the medium and selected depending on whether nitrate or ammonia is expected to be taken up from the medium.

Any suitable pH indicator may be used with the methods and assays of the present invention so long as the pH indicator is not phytotoxic and a change in pH can be detected over the desired range, for example, within a certain a pH range. Exemplary pH indicators useful in the methods and assays of the present invention include, but are not limited to bromophenol red (pH transition interval: from about 5.2 to about 7.0), chlorophenol red (pH transition interval: from about 5.0 to about 6.6), bromocresol purple (pH transition interval: from about 5.2 to about 6.8), and fluorescein (pH transition interval: from about 5.0 to about 9.0) or derivatives thereof such as Oregon Green dyes, a fluorinated analog of fluorescein that is pH sensitive (pH transition interval: from about 4.0 to about 6.0). In one aspect, the pH indicator may have a specific pH range, for example, from a pH of about 3.0 to about 9.0. In another aspect, the pH indicator may have a specific pH range from about 4.0 to about 7.0

In one aspect, the pH indicator is fluorescein which has the advantages of being both highly absorptive and fluorescent across a broad pH range. In one aspect, the fluorescein pH indicator can be measured at its characteristic pH-dependent absorption at 590 nanometers or its fluorescence excitation and emission spectra, for example, at wavelengths of about 420 nm and about 530 nm respectively.

In another aspect, pH indicators useful in the present invention change color or fluorescence intensity at their pH transition range, for example, with chlorophenol red or bromophenol red, the color changes from yellow→red or pink→purple; with bromocresol purple the color changes from purple→yellow in acidic conditions, fluorescein increases as pH increases. The color change or change in fluorescence of the pH indicator is indicative of a change in the pH of the medium and can be monitored using any suitable technique.

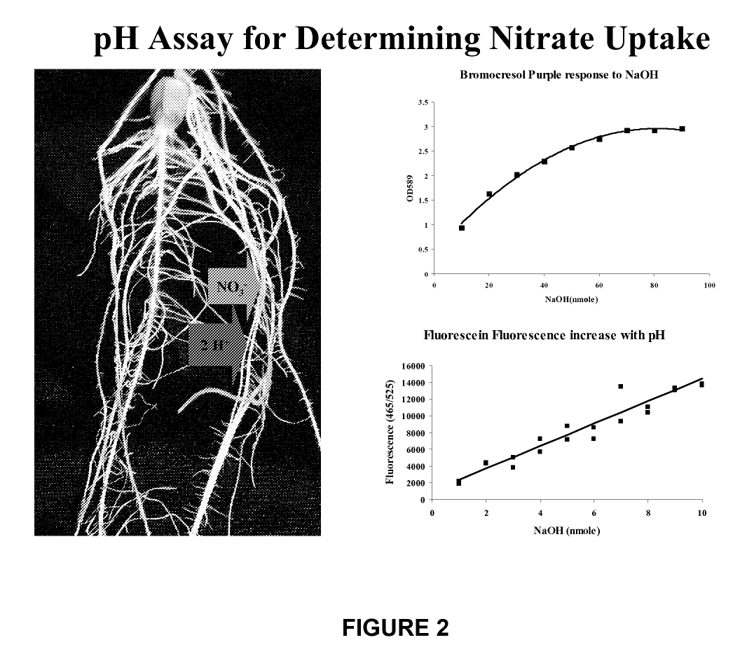

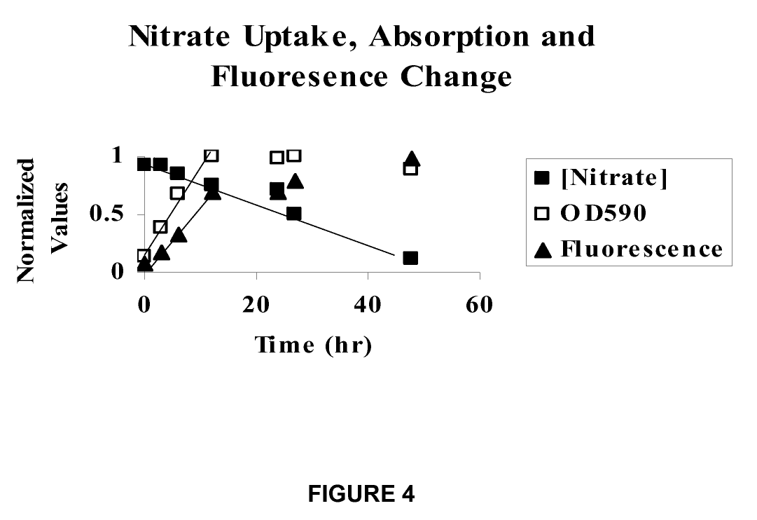

Accordingly, the methods and assays described herein include the determination of the pH in the plants' medium using one or more pH indicators to detect a change in the pH of the medium of the plants as the plants take up nitrogen. The pH of medium is known to be higher (more basic) after nitrate uptake and lower (more acidic) after ammonia uptake from the plants' medium. In one aspect, the invention provides for obtaining a sample of medium from one or more plants and screening the plants for nitrogen uptake using a pH indicator. The medium may be assayed using any suitable technique that detects a change in pH. In one aspect, the change in pH is detected using an optical technique, including visual observation of color change of the medium, fluorescence-based techniques and/or those techniques based on absorbance, including without limitation a scanning device, a fluorometer, a microplate reader, and a spectrofluorometer. For example, the absorption of the pH indicator may be used to determine a change in the pH of the medium. As discussed previously, as nitrate is taken up from the medium, the medium becomes more basic and the pH increases. When the pH of the medium having nitrate increases, the pH indicator changes color (and the color of the medium) and the absorption increases. Absorption of the medium may be determined at the appropriate wavelength for the individual pH indicator but typically the absorption is measured at a wavelength of 590 nm. See, FIGS. 2 and 4. Similarly, the fluorescence of the pH indicator may be used to determine a change in the pH of the medium. As the nitrate content decreases from the medium and is taken up by the plant, the pH of the medium becomes more basic and fluorescence increases. See, FIGS. 2 and 4. Conversely, as ammonia is taken up from a medium, the medium becomes more acidic and the pH decreases and absorption decreases. When the pH of the medium decreases as ammonia is taken up, pH indicators that fluoresce have decreased fluorescence. Fluorescence of the medium may be determined by exciting the pH indicator in the medium at the appropriate wavelength and detecting the emission spectra at the appropriate wavelength, for example, with respect to fluorescein the appropriate wavelengths are 420 nm and 530 nm respectively. Thus, the amount of absorbance or fluorescence may be measured and correlated to a concentration of the nitrate or ammonia remaining in the medium. In another aspect, the absorption and/or fluorescence data may be measured over a period of time to determine a rate of nitrogen uptake for each plant, for example, nitrate or ammonia uptake.

The pH of the medium may be determined and compared with the initial starting pH of the plant or control's medium to determine a change in pH. In one aspect, the changes in the pH for each plant may be compared to the change in pH of the medium of the control or of any plant of interest. For example, the pH's of the mediums for multiple plants can be monitored at various time points in growth and compared to other plants. Change in pH for individual plants may be determined and compared to other plants. The difference or change in pH over a period of time may be compared to the change in pH of the medium of the control plant or to another plant, for example, to determine which plants take up more nitrogen and/or at a faster rate relative to another plant.

The change in the pH of the medium may vary depending on the plants being screened, the plants' genetic makeup and the level of nitrogen present in the medium. The change in the pH between or among the medium of the plants being assayed and/or a control plant can be narrow or broad. In one aspect, the change in pH of the medium of the subject plant is determined and compared to the change in pH of the control plant. In one aspect, the amount of nitrate or ammonium remaining in the medium is determined and compared to the control plant.

Accordingly, the pH of the medium can be used to monitor nitrogen uptake of a plant, to identify a plant that has increased nitrogen uptake as compared to another plant, or to screen candidate polynucleotides that modulate nitrogen uptake of a plant. By comparing the changes in the pH of the medium of one plant to another, one can identify a plant with an increased nitrogen uptake or a plant that is more efficient at nitrogen uptake relative to another plant. A plant may be identified as a plant that has increased nitrogen uptake or a plant that is more efficient at nitrogen uptake relative to another plant using, for example, qualitative, quantitative, or statistical evaluation.

In one aspect, the changes in pH between or among plants, e.g., a subject and a control plant, may be evaluated using visual inspection of the medium. For example, one may determine whether a plant took up more nitrogen or at a faster rate than another by observing the color of the mediums and comparing them, e.g., to determine if the medium changed color, indicating a pH change. If the mediums of the subject and another plant undergoing comparison are the same hue, the hue of the medium may be evaluated to determine if one medium appears to be darker or lighter than the other. Such knowledge for determining changes in pH and ascertaining which color represents a more alkaline or acidic composition are within the knowledge of one skilled in the art. Thus, with respect to plants exposed to medium having nitrate, a medium that is observed to be more basic than the medium of another plant indicates that the plant with more basic medium may be considered to have greater nitrate uptake or be more efficient at nitrate uptake than the other plant. Conversely, with respect to plants exposed to medium having nitrate, a medium that is observed to be more acidic than the medium of another plant indicates that the plant with more acidic medium may be considered to have lower nitrate uptake or be less efficient at nitrate uptake than the other plant. Accordingly, with respect to plants exposed to medium having ammonia, a medium that is observed to be more acidic than the medium of another plant indicates that the plant with more acidic medium may be considered to have greater ammonia uptake or be more efficient at ammonia uptake than the other plant. Conversely, with respect to plants exposed to medium having ammonia, a medium that is observed to be more basic than the medium of another plant indicates that the plant with more basic medium may be considered to have lower ammonia uptake or be less efficient at ammonia uptake than the other plant. Thus, a calorimetric determination of the medium may made to determine whether a plant has greater nitrate uptake (or ammonia uptake as appropriate) compared to another plant using the methods and assays of the present invention.

In another aspect, the changes in pH between or among plants, e.g. a subject and a control plant, may be evaluated using absorbance or fluorescence data. The change in absorbance or fluorescence for the medium of each plant may be determined and compared between or among plants, e.g., a control plant, to determine the differences. With respect to plants exposed to medium having nitrate, a plant having a greater or greatest difference in absorbance or fluorescence may be considered to have greater nitrate uptake or to be more efficient at nitrate uptake than another plant. With respect to plants exposed to medium having ammonia, a plant having a greater or greatest difference in absorbance or fluorescence may be considered to have greater ammonia uptake or to be more efficient at ammonia uptake than another plant.

In another aspect, plants may be considered to be more efficient at nitrate or ammonia uptake between or among other plants if the difference in pH change is statistically significant. Statistically significant refers to having a p-value <0.05. The term “p-value <0.05” refers to the chance of a result being obtained, at random, as less than 5 times in 100. The differences in pH change may be determined using absorption or fluorescence data in combination with a statistical method, such as a t-test. The t-test or any other suitable formula may be used to obtain a p-value. Plants that have p-values <0.05 with respect to a change in absorbance or fluorescence are indicative of plants that are more efficient at nitrogen uptake.

Additionally, the amount of nitrate or ammonia remaining in the medium may be determined by any number of routine protocols, such as the one by described in Examples 4 and 5 respectively.

Using methods or assays of the present invention, one skilled in the art would be able to screen thousands of different plants, for example, for their ability to uptake nitrate or ammonia. The methods of the present invention are useful for a variety of applications. As discussed, the methods and assays of the present invention permit the identification of plants with increased nitrogen uptake or efficiency. These plants may be additionally screened for their ability to increase the yield or biomass of a plant as compared to a control.

The present invention provides for a high throughput assay for screening a plurality of plants to identify a plant with increased nitrogen uptake, for example, a plant having a candidate polynucleotide suspected of increasing nitrogen uptake of a plant. In one aspect, the method includes determining the pH of a medium comprising nitrogen and a pH indicator. Any suitable pH indicator as described herein may be used with the assay.

In one aspect, the source of nitrogen is nitrate or ammonia. The plants are exposed to the medium for a time sufficient for the nitrogen to be taken up by the plants. The pH of the medium may be measured for an initial starting pH and again determined at a later point in time, for example, after a sufficient amount of time for the nitrogen to be taken up by the plants has passed. In one aspect, each plant may be placed in a well in a microtiter plate having a plurality of wells comprising medium. In another aspect of the assay, each plant may be placed in a pot. In one aspect, the plant is immersed in a medium comprising the source of nitrogen, such as potassium nitrate. In one aspect, the medium is a nutrient solution such as modified Hoagland's solution (Hoagland and Amon, 1938). In another aspect, the medium is PHYTAGEL® agar substitute gelling agent. In one aspect, the plants to be screened are seeds. The seeds may be stratified and exposed to cycles of light and dark to promote germination. The status of the seed may be evaluated for germination and the medium evaluated for a change in pH, for example, the change in color of the medium using visual inspection or another optical detection method. The plant may be removed from the well to facilitate pH determination of the medium. The assay further includes administering another pH indicator, such as fluorescein, to the medium and determining the fluorescence of the pH indicator to determine the concentration of the remaining nitrogen in the medium. In another aspect, the assay includes correlating the change in pH of the medium to a concentration of nitrate or ammonia in the medium.

The change in pH of the plants' mediums identifies a polynucleotide that modifies nitrogen uptake of a plant. As described herein, an increase in the pH (increased alkalinity), absorbance, or fluorescence of the medium with nitrate indicates increased nitrate uptake, a decrease in the pH (increased acidity), absorbance, or fluorescence of the medium with ammonia indicates increased ammonia uptake. Thus, an increase in the pH of medium having nitrate identifies the polynucleotide as a candidate polynucleotide for use in increasing nitrate uptake of a plant. A decrease of the pH of medium having ammonia identifies the polynucleotide as a candidate polynucleotide for use in increasing ammonia uptake of a plant. Plants containing the identified polynucleotide may be further evaluated for improving the yield or biomass of the plant having the candidate polynucleotide. The plant having the candidate polynucleotide may be compared to one or more plants that do not contain the candidate polynucleotide.

The assay may include any aspect described herein with respect to the methods of the present invention, for example, the use of various pH indicators, sources of nitrogen, plants and medium and various optical detection techniques.

This invention can be better understood by reference to the following non-limiting examples. It will be appreciated by those skilled in the art that other embodiments of the invention may be practiced without departing from the spirit and the scope of the invention as herein disclosed and claimed.

### EXAMPLES

The present invention is further defined in the following Examples, in which parts and percentages are by weight and degrees are Celsius, unless otherwise stated. The disclosure of each reference set forth herein is incorporated herein by reference in its entirety.

### Example 1

### Screen to Identify Lines with Improved Nitrate Uptake

For each overexpressor line, twelve T2 plants are sown on 96 well micro titer plates containing 2 mM MgSO₄, 0.5 mM KH₂PO₄, 1 mM CaCl₂, 2.5 mM KCl, 0.15 mM Sprint 330, 0.06 mM FeSO₄, 1 μM MnCl₂ 4H₂O, 1 μM ZnSO₄.7H₂O, 3 μM H₃BO₃, 0.1 μM NaMoO₄, 0.1 μM CuSO₄.5H₂O, 0.8 mM potassium nitrate, 0.1% sucrose, 1 mM MES, 200 μM bromophenol red and 0.40% Phytagel™ (pH assay medium). The pH of the medium is so that the color of bromophenol red, the pH indicator dye, is yellow.

Four lines are plated per plate, and the inclusion of 12 wild-type individuals and 12 individuals from a line that has shown an improvement in nitrate uptake (positive control) on each plate makes for a total of 72 individuals on each 96 well micro titer plate A web-based random sequence generator was used to determine the order of the lines on each plate. Seeds are not plated in Row A or Row H on the 96 well micro titer plate. Four plates are plated for each experiment, resulting in a maximum of 48 plants per line analyzed. Plates are kept for three days in the dark at 4° C. to stratify seeds, and then placed horizontally for six days at 22° C. light and dark. Photoperiod is sixteen hours light; eight hours dark, with an average light intensity of ˜200 mmol/m²/s. Plates are rotated and shuffled within each shelf. At day eight or nine (five or six days of growth), seedling status is evaluated by recording the color of the medium as pink, peach, yellow or no germination. Then the plants and/or seeds are removed from each well. Each medium plug is transferred to 1.2 ml micro titer tubes and placed in the corresponding well in a 96 well deep micro titer plate. An equal volume of water containing 2 μM flourescein is added to each 1.2 ml micro titer tube. The plate is covered with foil and autoclaved on liquid cycle. Each tube is mixed well, and an aliquot is removed from each tube and analyzed for amount of nitrate remaining in the medium. If t-test shows that a line is significantly different (p<0.05) from wild-type control, the line is then considered a validated improved nitrate uptake line.

### Example 2

Maize plants were planted in TURFACE® MVP. (Profile Products LLC (Buffalo Grove, Ill.)) contained in a 3.5 inch square pot and watered with nutrients (Table 1) after dilution through a siphoning mechanism. Plant and pots were submerged in 1 liter of 16× dilution of the 1 mM KNO3 nutrient solution containing 100 μM bromocresol purple and 0.5 μM fluorescein with the pH adjusted to 5.2. All containers were aerated. At regular intervals 500 μl aliquots were removed and the optical density at 590 nM, fluorescence (excitation 420 nM, emission 530 nM) and nitrate concentration determined. These were plotted with time and compared to loss of nitrate from the medium.

TABLE 1
Components of concentrated plant nutrient solutions.

| Ingredient | 1 mM KNO₃ | | 2 mM KNO₃ | |
|---|---|---|---|---|
| KH₂PO₄ | 11 | g | 11 | g |
| CaCl₂ | 47 | g | 47 | g |
| KNO₃ | 32.3 | g | 64.6 | g |
| KCl | 71 | g | 47.7 | g |
| MgSO₄ | 38.4 | g | 38.4 | g |
| Sprint330 | 32 | g | 32 | g |
| 10x Micros | 16 | ml | 16 | ml |

/20 liter
H₂SO₄ added 1.5-2 ml/10 liters, as required, to maintain final nutrient pH
at 5-6.

| 10X Micronutrients | mg/liter |
|---|---|
| 30 mM H₃BO₃ | 1854 |
| 10 mM MnCl₂•4H₂O | 1980 |
| 10 mM ZnSO₄•7H₂O | 2874 |
| 1 mM CuSO₄•5H₂O | 250 |
| 1 mM H₂MoO₄•H₂O | 242 |

### Example 3

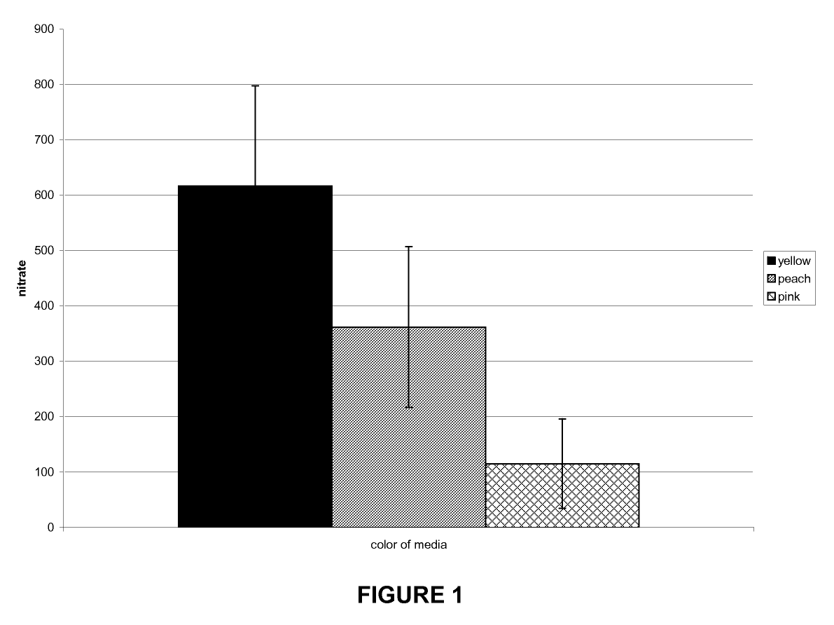

When an individual Arabidopsis plant is grown in each well with medium containing bromophenol red, a pH indicator dye, and 0.8 mM KNO3, the medium will change from yellow to pink, indicating the pH of the medium is more basic. When this medium from each well is analyzed to determine the amount of nitrate remaining in the medium, the majority of the wells classified as pink have the least amount of nitrate remaining in the medium while the majority of the wells classified as yellow have the greatest amount of nitrate remaining in the medium (FIG. 1), indicating that the change in pH detected by the pH indicator dye may be used to monitor nitrate transport.

Plants use both a high-affinity transport systems and low-affinity transport systems to take up nitrate from the rhizosphere. The first nitrate transporter identified in higher plants was AtNRT1.1 (Tsay, et al., 1993). This transporter was originally classified as a low-affinity nitrate transporter; however, further characterization revealed that it is a dual-affinity nitrate transporter (Liu, et al., 1999 and Wang, et al, 1998). This transporter switches from low-affinity to high-affinity by using a phosphorylation mode of action (Liu and Tsay, 2003). There are 7 high-affinity nitrate transporters in Arabidopsis with the most studied ones being AtNRT2.1 and AtNRT2.2. A reduction in high-affinity nitrate transport was first detected in a mutant in which both AtNrt2.1 and AtNrt2.2 genes were disrupted (Filleur, et al., 2001). Recently, AtNRT2.1 was shown to be the major contributor to the inducible high affinity transport system (iHATS) (Li, et al., 2007). If AtNrt2.1 is disrupted, iHATS is reduced up to 72% while constitutive high affinity transport systems (cHATS) and low affinity nitrate transport systems (LATS) are not significantly affected. This reduction in iHATS results in a reduction of the shoot to root ratio. If two mutants Atnrt1.1 and Atnrt2.1 were analyzed using the pH indicator dye nitrate uptake assay, one expectation is that the color of the medium would change slower for each of the mutants when compared to wild-type plants. Nitrate analysis of the medium would reveal significantly more nitrate remaining in the medium in wells that contained the mutants when compared to medium in wells containing wild-type plants. However, another expectation is that no significant difference would be detected between mutants and wild-type since gene compensation has been described for AtNrt1.1 and AtNrt2.1 (Munos, et al., 2004) and for AtNrt2.1 and AtNrt2.2 (Li, et al., 2007). When Atnrt1.1 and Atnrt2.1 were individually analyzed using the pH indicator dye assay, the majority of the wells remained yellow while the wells containing wild-type plants were beginning to change color. Nitrate analysis of the medium revealed that both Atnrt1.1 and Atnrt2.1 took up significantly less nitrate from the medium than wild-type plants when grown in the presence of the pH indicator dye. When multiple plants are grown in the same well, the majority of the medium changes from yellow to pink faster than wells that only contained a single plant. Nitrate analysis of this medium revealed that wells that had three plants had significantly less nitrate remaining in the medium than wells that had either two plants or one plant. Furthermore, wells that contained two plants had significantly less nitrate remaining in the medium than wells that contained only a single wild-type plant.

### Example 4

### Nitrate Determinations

### Stock Solutions.

20 mM NaNO₃ stock solution
20 mM NaNO₂ stock solution (optional)
8 mM NADP stock
600 mM glucose-6-phosphate
100 mM cysteine—free base—prepare fresh each day (12.12 mg/ml)
Glucose-6-phosphate dehydrogenase—G6PDH (Sigma G-8529) 200 units dissolved in 0.25 ml 25 mM Tris-HCl pH=7.5—50% glycerol
Nitrate Reductase—NR (Sigma N-7265) 10 units dissolved in 720 μl 25 mM Tris-HCl
pH=7.5—50% glycerol
1M Tris-HCl pH=7.5
25 mM Tris-HCl pH=7.5—50% glycerol
SA—H₃PO₄—NEDA—1% sulfanilamide in 2M H₃PO₄—0.02% N-(1-naphthyl)ethylenediamine. Store sealed in darkened vessel.

### Acetonitrile

Assay mix for 100 nitrate assays

1.5 ml 1M Tris-HCL pH=7.5

100 μl—600 mM glucose-6-phosphate

100 μl—100 mM cysteine

15 μl 8 mM NADP

10 μl G6PDH

100 μl NR

175 μl—water

### Procedure.

Samples containing nitrate in the concentrations from 20 to 2000 μM are added to individual wells of a 96 well plate and brought up to 100 μl with water.

Prepare 2 sets of standards by diluting 20 mM NaNO₃ 1:10 and 1:100 to form 2 mM and 0.2 mM standard solutions, respectively. The nitrate standard curve is prepared by adding 0, 10, 20, 30, 40, 50, 60, 70, 80, 90 and 100 μl of each standard solution to duplicate wells of a 96 well plate. Water is added to each to bring the volume up to 100 μl.

Add 20 μl of the assay mix to all sample and standard wells. Mix gently and incubate for 1-2 hr at room temperature (25-30° C.). During this time determine fluorescein concentration by measuring fluorescence (Ex 480, Em 530). This measurement will be used to correct for deviations due to sample handling. Add 50 μl of acetonitrile. Add 100 μl SA-H₃PO₄—NEDA. Allow 30 min for complete color development and read the optical density at 540 nm. Remove 25 μl of the higher range standards and add to an additional 200 μl SA-H₃PO₄—NEDA and determine optical density at 540 nm. Any sample that is out of range of the lower standard curve (OD⁵⁴⁰>2.8) treat similarly as the higher range standards, remove 25 μl and add to an additional 200 μl SA-H₃PO₄—NEDA. Use the upper range standard curve for these determinations.

### Example 5

### Ammonia Quantification

### Stock Solutions

1M Borate Buffer pH=9.5 (3.09 g H₃BO₄+1 g NaOH)
1 mM NH₄Cl make up fresh daily
OPA stock solution—50 mg OPA (o-phthaldialdehyde Sigma # P0657) in 1.5 ml methanol plus 11 ml of 0.4 M NaBO₄ pH=9.5, 1% SDS. Prepare fresh, weekly.
OPA—working solution 1 ml OPA stock+5 uL mercaptoethanol. Prepare fresh, daily.

### Procedure

Add sample containing concentrations of ammonia from 100 to 1000 uM into separate wells of a 96 well plate. Bring the volume up to 200 ul with and add 50 μl OPA working solution. Read fluorescence (360 Ex/528 Em) immediately using a standard curve of NH₄Cl from 10-100 μl mM NH₄Cl. Read the standard curve first and last and use the average as the standard curve. There is a good linear fit r=0.992. After 1.5 hr there is a good linear fit but a better quadratic fit (r=1.00).

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

What is claimed is:

1. A method to monitor nitrogen uptake of a plant comprising:
determining the pH of a medium comprising a source of nitrogen and a pH indicator;
exposing a plant to nitrogen in the medium for a time sufficient for the nitrogen to be taken up by the plant; and
optically detecting a change in the pH of the medium of the plant.

2. The method of claim 1 wherein the source of nitrogen is ammonia or nitrate.

3. The method of claim 2 wherein the source of nitrate is potassium nitrate.

4. The method of claim 2 wherein the source of ammonia is ammonium citrate or ammonium succinate.

5. The method of claim 1 wherein the plant is a non-transformed plant, an inbred, a hybrid or a plant transformed with a polynucleotide.

6. The method of claim 1 wherein the plant is a plant treated with a mutagen.

7. The method of claim 1 wherein a plurality of plants are screened.

8. The method of claim 1 wherein the medium is agar, soil, or a solution.

9. The method of claim 8 wherein the agar is PHYTAGEL® agar substitute gelling agent.

10. The method of claim 8 wherein the solution is a nutrient solution.

11. The method of claim 1 wherein the pH indicator is not phytotoxic.

12. The method of claim 1 wherein the pH indicator is a dye.

13. The method of claim 1 wherein the pH indicator has a pH range from about 3.0 to about 9.0.

14. The method of claim 13 wherein the pH indicator has a pH range from about 4.0 to about 7.0.

15. The method of claim 1 wherein the pH indicator comprises bromophenol red, chlorophenol red, bromocresol purple and fluorescein or derivatives thereof.

16. The method of claim 1 further comprising optically detecting the change in pH of the medium by color change of the medium.

17. The method of claim 1 further comprising optically detecting the change in pH of the medium using visual inspection, a scanning device, a fluorometer, a microplate reader, and a spectrofluorometer.

18. The method of claim 1 further comprising optically detecting the change in pH of the medium by measuring the absorption of the medium.

19. The method of claim 18 further comprising measuring the absorption of the medium at a wavelength of approximately 590 nanometers.

20. The method of claim 18 wherein the amount of absorption is correlated to the amount of nitrogen remaining in the medium.

21. The method of claim 20, wherein the source of nitrogen is nitrate, an increase in the absorption of the medium is indicative of increased nitrate uptake.

22. The method of claim 20, wherein the source of nitrogen is nitrate, a decrease in the absorption of the medium is indicative of decreased nitrate uptake.

23. The method of claim 20, wherein the source of nitrogen is ammonia, a decrease in the absorption of the medium is indicative of increased ammonia uptake.

24. The method of claim 20, wherein the source of nitrogen is ammonia, an increase in the absorption of the medium is indicative of decreased ammonia uptake.

25. The method of claim 1 further comprising optically detecting the change in pH of the medium by measuring the fluorescence of the medium.

26. The method of claim 1 further comprising adding a pH indicator that fluoresces to the medium and determining the fluorescence.

27. The method of claim 25 further comprising determining the fluorescence of the medium by exciting the pH indicator in the medium at an appropriate wavelength and detecting emission at an appropriate wavelength.

28. The method of claim 15 wherein the pH indicator that fluoresces is fluorescein or a derivative thereof.

29. The method of claim 25 wherein the amount of fluorescence is correlated to the amount of nitrogen remaining in the medium.

30. The method of claim 29, wherein the source of nitrogen is nitrate, an increase in the fluorescence of the medium is indicative of increased nitrate uptake.

31. The method of claim 29, wherein the source of nitrogen is nitrate, a decrease in the fluorescence of the medium is indicative of decreased nitrate uptake.

32. The method of claim 29, wherein the source of nitrogen is ammonia, a decrease in the fluorescence of the medium is indicative of increased ammonia uptake.

33. The method of claim 29, wherein the source of nitrogen is ammonia, an increase in the fluorescence of the medium is indicative of decreased ammonia uptake.

34. The method of claim 1 further comprising comparing the change in pH of the medium to a change in pH of the medium of a second plant exposed to the same medium.

35. The method of claim 1 further comprising comparing the change in pH of the medium to a control.

36. The method of claim 1 further comprising comparing the rate of nitrate uptake for a first plant compared to a second plant.

37. The method of claim 1 further comprising comparing the rate of ammonia uptake for a first plant compared to a second plant.

38. The method of claim 1 further comprising analyzing the plant comprising a candidate polynucleotide for improved yield or biomass.

39. An assay for high throughput screening of candidate polynucleotides for use in modulating nitrogen uptake of a plant comprising:
determining the pH of a medium comprising a source of nitrogen and a pH indicator;
exposing a plant comprising a candidate polynucleotide to nitrogen in the medium for a time sufficient for the nitrogen to be taken up by the plant; and
optically detecting a change in the pH of the medium of the plant, whereby the change in pH identifies a polynucleotide that modifies nitrogen uptake of a plant.

40. The assay of claim 39 wherein the source of nitrogen is ammonia or nitrate.

41. The method of claim 40 wherein the source of nitrate is potassium nitrate.

42. The method of claim 40 wherein the source of ammonia is ammonium citrate or ammonium succinate.

43. The assay of 39 wherein the medium is agar, soil, or a solution.

44. The assay of 43 wherein the agar is PHYTAGEL® agar substitute gelling agent.

45. The assay of claim 43 wherein the solution is a nutrient solution.

46. The assay of claim 39 wherein the plant is a non-transformed plant, an inbred, a hybrid or a plant transformed with a polynucleotide.

47. The assay of claim 39 wherein the plant is a plant treated with a mutagen.

48. The assay of claim 39 wherein a plurality of plants are screened.

49. The assay of claim 39 wherein the plant is a seed.

50. The assay of claim 49 wherein the seed is stratified.

51. The assay of claim 49 wherein the seed is exposed to cycles of light and dark.

52. The assay of claim 39 wherein each plant is placed in a well in a microtiter plate having a plurality of wells comprising medium.

53. The assay of claim 50 further comprising evaluating the status of the seed for germination.

54. The assay of claim 39 wherein each plant is placed in a pot.

55. The assay of claim 54 wherein each plant is immersed in a solution comprising the source of nitrogen.

56. The assay of claim 39 wherein the pH indicator is not phytotoxic.

57. The assay of claim 39 wherein the pH indicator is a dye.

58. The assay of claim 39 wherein the pH indicator has a pH range from about 3.0 to about 9.0.

59. The assay of claim 58 wherein the pH indicator has a pH range from about 4.0 to about 7.0.

60. The assay of claim 39 wherein the pH indicator comprises bromophenol red, chlorophenol red, bromocresol purple and fluorescein or derivatives thereof.

61. The assay of claim 39 further comprising optically detecting the change in pH of the medium by color change of the medium.

62. The assay of claim 39 further comprising optically detecting the change in pH of the medium using visual inspection, a scanning device, a fluorometer, a microplate reader, and a spectrofluorometer.

63. The assay of claim 39 further comprising optically detecting the change in pH of the medium by measuring the absorption of the medium.

64. The assay of claim 63 further comprising measuring the absorption of the medium at a wavelength of approximately 590 nanometers.

65. The assay of claim 63 wherein the amount of absorption is correlated to the amount of nitrogen remaining in the medium.

66. The assay of claim 65, wherein the source of nitrogen is nitrate, an increase in the absorption of the medium is indicative of increased nitrate uptake.

67. The assay of claim 65, wherein the source of nitrogen is nitrate, a decrease in the absorption of the medium is indicative of decreased nitrate uptake.

68. The assay of claim 65, wherein the source of nitrogen is ammonia, a decrease in the absorption of the medium is indicative of increased ammonia uptake.

69. The assay of claim 65, wherein the source of nitrogen is ammonia, an increase in the absorption of the medium is indicative of decreased ammonia uptake.

70. The assay of claim 39 further comprising optically detecting the change in pH of the medium by measuring the fluorescence of the medium.

71. The assay of claim 39 further comprising adding a pH indicator that fluoresces to the medium and determining the fluorescence.

72. The assay of claim 70 further comprising determining the fluorescence of the medium by exciting the pH indicator in the medium at an appropriate wavelength and detecting emission at an appropriate wavelength.

73. The assay of claim 60 wherein the pH indicator that fluoresces is fluorescein or a derivative thereof.

74. The assay of claim 70 wherein the amount of fluorescence is correlated to the amount of nitrogen remaining in the medium.

75. The assay of claim 74, wherein the source of nitrogen is nitrate, an increase in the fluorescence of the medium is indicative of increased nitrate uptake.

76. The assay of claim 74, wherein the source of nitrogen is nitrate, a decrease in the fluorescence of the medium is indicative of decreased nitrate uptake.

77. The assay of claim 74, wherein the source of nitrogen is ammonia, a decrease in the fluorescence of the medium is indicative of increased ammonia uptake.

78. The assay of claim 74, wherein the source of nitrogen is ammonia, an increase in the fluorescence of the medium is indicative of decreased ammonia uptake.

79. The assay of claim 39 further comprising comparing the change in pH of the medium to a change in pH of the medium of a second plant exposed to the same medium.

80. The assay of claim 39 further comprising comparing the change in pH of the medium to a control.

81. The assay of claim 39, wherein the source of nitrogen is nitrate, further comprising comparing the rate of nitrate uptake for a first plant compared to a second plant.

82. The assay of claim 39, wherein the source of nitrogen is ammonia, further comprising comparing the rate of ammonia uptake for a first plant compared to a second plant.

83. The assay of claim 39 further comprising analyzing the plant comprising a candidate polynucleotide for improved yield or biomass.

84. The assay of claim 83 further comprising analyzing the plant comprising the candidate polynucleotide for improved yield or biomass as compared to a control that does not contain the candidate polynucleotide.
